(19) United States
(12) Patent Application Publication
(10) Pub. No.: US 2023/0363629 A1
(43) Pub. Date: Nov. 16, 2023

(54) IMAGE FORMING APPARATUS

(71) Applicant: Tamron Co., Ltd., Saitama (JP)

(72) Inventors: Naoki YAMASHITA, Saitama (JP); Akira ONODA, Saitama (JP); Toshiya SEGAWA, Saitama (JP)

(73) Assignee: Tamron Co., Ltd., Saitama (JP)

(21) Appl. No.: 18/134,870

(22) Filed: Apr. 14, 2023

(30) Foreign Application Priority Data
May 13, 2022 (JP) 2022-079762

(51) Int. Cl. A61B 1/04; A61B 1/06; G06T 5/50

(52) U.S. Cl. CPC A61B 1/043; A61B 1/0638; G06T 5/50

(57) ABSTRACT

An image forming apparatus includes a light source that irradiates an observation target with excitation light; an imaging part that separately receives short wavelength-side NIR light and SWIR light from the observation target; and an image processing unit that generates a composite image having both a density of a short wavelength-side NIR light image and a definition of an SWIR light image.

### CROSS-REFERENCE TO RELATED APPLICATIONS

This application is based on and claims the benefit of priority from Japanese Patent Application No. 2022-079762, filed on May 13, 2022, the entire contents of which are incorporated herein by reference.

### BACKGROUND OF THE INVENTION

### Technical Field

The present invention relates to an image forming apparatus.

### Related Art

Fluorescence imaging is known as a medical observation system that specifies whether there is a tumor in a biological tissue and a position of the tumor. The fluorescence imaging is a technique in which a fluorescent reagent is administered into a living body to be specifically accumulated in a tumor or the like in the living body, and then the fluorescent reagent is excited by light having a specific wavelength, so that fluorescence emitted by the fluorescent reagent is imaged to display as an image of the fluorescence. By detecting the fluorescence in the living body as described above, it is possible to ascertain whether there is a tumor and a position of a tumor.

Indocyanine green (ICG) is generally used as a medical fluorescent reagent. The ICG is excited by light having a wavelength included in a near-infrared (NIR) light region (750 to 850 nm), in which biological permeability is excellent, and emits fluorescence having a peak wavelength of about 835 nm included in the NIR region. It has been known that a wavelength region of fluorescence of ICG reaches a short wavelength infrared (SWIR) region on a longer wavelength side (900 to 1600 nm). A component of the fluorescence of ICG is less affected by scattering of light by a living body in the SWIR region than in the NIR region (835 nm). Therefore, it is expected to observe a deep part of the living body at 1 cm or more below the skin or acquire a high-resolution image.

As a technique for detecting fluorescence in a short wavelength infrared region, there has been known a technique for detecting fluorescence of ICG at 900 nm or more using an SWIR sensor (for example, see JP 2019-513229 A). In addition, as a technique for detecting fluorescence in a short wavelength infrared region, there has been known a technique for detecting fluorescence of an inorganic fluorescent substance such as Yb, Nd, or Er using an SWIR sensor (for example, see JP 2013-162978 A).

The light having a wavelength near the peak of the fluorescence of ICG has a high brightness, but is generally likely to be scattered by a biological tissue when generated at a deeper site of a living body, i.e., at a site of about several mm to 1 cm below the skin, and as a result, a corresponding light image has a low resolution.

On the other hand, the fluorescence of ICG in the SWIR region is less affected by scattering of light by a living body than the fluorescence having a wavelength near the peak, but a corresponding light image has a low brightness. In addition, the SWIR range also includes water absorption. Therefore, in a case where fluorescence is generated at a deeper site of a living body, a corresponding fluorescence image has a high resolution, but its contrast deteriorates.

As a way of increasing a contrast of an image, an increase in exposure time or amount of excitation light has been known. However, the increase in exposure time may also increase background accordingly, and the increase in amount of excitation light may increase the reflection of the excitation light or the influence of the excitation light on the living body.

An object of an aspect of the present invention is to provide a new technology capable of acquiring an image having both a feature of a high-brightness image and a feature of a high-resolution image.

### SUMMARY OF THE INVENTION

An image forming apparatus according to an aspect of the present invention for solving the aforementioned problems includes: an excitation light source that irradiates an observation target with excitation light; an imaging part that receives first infrared light and second infrared light split from light from the observation target irradiated with the excitation light, the first infrared light including light having a wavelength in a short wavelength infrared region, and the second infrared light including light having a wavelength in a wavelength region on a shorter wavelength side than the short wavelength infrared region; and an image processing unit that generates a composite image by combining a first image and a second image, the first image indicating a boundary of a specific region corresponding to the first infrared light received by the imaging part, and the second image including a specific region having an image density corresponding to the second infrared light received by the imaging part.

According to an aspect of the present invention, it is possible to provide a new technology capable of acquiring an image having both a feature of a high-brightness image and a feature of a high-resolution image.

### BRIEF DESCRIPTION OF THE DRAWINGS

FIG. 1 is a diagram schematically illustrating a functional configuration of an image forming apparatus according to a first embodiment of the present invention;

FIG. 2 is a block diagram illustrating a functional configuration of an image processing unit of the image forming apparatus according to the first embodiment of the present invention;

FIG. 3 is a diagram illustrating photographs representing a short wavelength-side NIR image obtained by directly imaging a test specimen included in an observation target example when is irradiated with excitation light, a short wavelength-side NIR image of the observation target example, and an SWIR image of the observation target example, respectively, in the first embodiment of the present invention;

FIG. 4 is a flowchart illustrating an example of an image forming process according to the first embodiment of the present invention;

FIG. 5 is a diagram illustrating an example of a histogram of an SWIR image according to the first embodiment of the present invention;

FIG. 6 is a diagram illustrating an example of a binarized SWIR image according to the first embodiment of the present invention;

FIG. 7 is a diagram illustrating an example of a contour of a binarized SWIR image and an example of a mask image formed based on the contour according to the first embodiment of the present invention;

FIG. 8 is a diagram illustrating an example of a composite image obtained by superimposing a mask image on a short wavelength-side NIR image according to the first embodiment of the present invention;

FIG. 9 is a diagram schematically illustrating a functional configuration of an image forming apparatus according to a second embodiment of the present invention;

FIG. 10 is a diagram illustrating an example of a relationship of a focus position with each wavelength of a focus shift correction lens in the image forming apparatus according to the second embodiment of the present invention;

FIG. 11 is a diagram schematically illustrating a functional configuration of an image forming apparatus according to a third embodiment of the present invention;

FIG. 12 is a diagram schematically illustrating a configuration of an NIR-SWIR filter according to the third embodiment of the present invention;

FIG. 13 is a diagram schematically illustrating a functional configuration of an image forming apparatus according to a fourth embodiment of the present invention;

FIG. 14 is a diagram schematically illustrating a configuration of a VIS-NIR filter according to the fourth embodiment of the present invention;

FIG. 15 is a diagram schematically illustrating a functional configuration of an image forming apparatus according to a fifth embodiment of the present invention;

FIG. 16 is a diagram schematically illustrating a functional configuration of an image forming apparatus according to a sixth embodiment of the present invention;

FIG. 17 is a diagram schematically illustrating a functional configuration of an image forming apparatus according to a seventh embodiment of the present invention;

FIG. 18 is a diagram schematically illustrating a configuration of an SWIR-SWIR filter according to the seventh embodiment of the present invention;

FIG. 19 is a diagram schematically illustrating a functional configuration of an image forming apparatus according to an eighth embodiment of the present invention;

FIG. 20 is a diagram schematically illustrating a configuration of a VIS-NIR-SWIR filter according to the eighth embodiment of the present invention;

FIG. 21 is a block diagram illustrating a functional configuration of an image processing unit of the image forming apparatus according to the eighth embodiment of the present invention;

FIG. 22 is a diagram illustrating a timing chart for explaining an example of an operation of the image forming apparatus according to the eighth embodiment of the present invention;

FIG. 23 is a flowchart illustrating an example of image processing for forming a composite image by top-hat transformation;

FIG. 24 is a diagram schematically illustrating an original image in the image processing for forming a composite image by top-hat transformation;

FIG. 25 is a diagram schematically illustrating an image obtained by expanding the original image by top-hat transformation;

FIG. 26 is a diagram schematically illustrating an image of a boundary portion extracted in the image processing for forming a composite image by top-hat transformation;

FIG. 27 is a diagram schematically illustrating a superimposition image of a boundary portion image and a second image in the image processing for forming a composite image by top-hat transformation;

FIG. 28 is a flowchart illustrating an example of image processing for forming a composite image by wavelet transformation;

FIG. 29 is a diagram schematically illustrating an original image in the image processing for forming a composite image by wavelet transformation; and

FIG. 30 is a diagram schematically illustrating frequency component images of the original image decomposed by the wavelet transformation.

DESCRIPTION OF THE EMBODIMENTS

### First Embodiment

Hereinafter, an embodiment of the present invention will be described in detail. In the present specification, the term “to” indicates a range including a smallest numerical value before the term “to” and a largest numerical value after the term “to”. An image forming apparatus according to an embodiment of the present invention will be described, assuming that an observation target is a person to be examined to whom ICG is administered in his/her body as a fluorescent reagent.

[Configuration of Image Forming Apparatus]

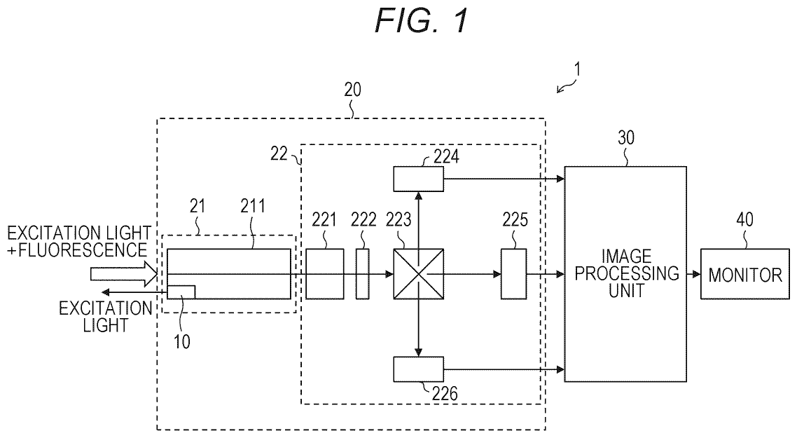

FIG. 1 is a diagram schematically illustrating a functional configuration of an image forming apparatus according to a first embodiment of the present invention. An image forming apparatus 1 has a function of imaging an observation target using visible light, and a function of imaging fluorescence emitted when ICG administered to the observation target is excited by irradiating excitation light such as near-infrared light. As illustrated in FIG. 1, the image forming apparatus 1 includes a light source 10, an imaging part 20, an image processing unit 30, and a monitor 40.

The light source 10 includes a visible light source and an excitation light source that emits near-infrared light for exciting ICG as excitation light. The excitation light source is, for example, a laser that generates light having a wavelength of 808 nm. The observation target is irradiated with the excitation light simultaneously with the visible light from the light source disposed at a distal end portion of a hard insertion part 21. Note that the simultaneous irradiation does not necessarily mean that the irradiation periods completely coincide with each other, and may mean that the irradiation periods at least partially overlap each other.

In addition, the excitation light is not limited to the light in the above-described wavelength range, and is appropriately determined depending on the type of fluorescent reagent.

The imaging part 20 includes a hard insertion part (probe) 21 and an imaging unit 22. The hard insertion part 21 is a portion to be inserted into a body of a person to be examined in a state where the ICG has been administered in advance, and has, for example, a cylindrical shape with a diameter of about 5-10 mm. The hard insertion part 21 includes a light source 10 and a first optical system 211. The first optical system 211 is, for example, an objective lens.

Note that the light source 10 may not be disposed at the distal end portion of the hard insertion part 21. For example, instead of the light source 10, the hard insertion part 21 may hold an optical fiber that guides light emitted from the light source 10.

The image forming apparatus 1 is configured so that light received by the hard insertion part 21 is guided to the imaging unit 22, while the hard insertion part 21 and the imaging unit 22 are detachably connected to each other. The configuration capable of guiding light may be, for example, a relay lens that transmits an image by light in a relay form or a configuration capable of realizing a method called pupil relay. In addition, the configuration capable of guiding light may be, for example, an optical fiber capable of transmitting image information, such as an image guide fiber.

The imaging unit 22 includes a second optical system 221, an excitation light cut filter 222, a dichroic prism 223, a first imaging unit 224, a second imaging unit 225, and a third imaging unit 226.

The second optical system 221 is, for example, an image forming lens. The excitation light cut filter 222 is an optical filter that reflects or absorbs only excitation light incident thereon for attenuation, and is, for example, a notch filter.

The dichroic prism 223 is a beam splitter that splits incident light into an SWIR light component, a short wavelength-side NIR light component, and a VIS light component in different directions, and is, for example, a cubic beam splitter having two kinds of optical thin films orthogonal to each other. The incident light is light from the person to be examined. The SWIR light component of the incident light is a light component having a wavelength in a short wavelength infrared region (e.g., 900 nm or more and 1600 nm or less) in the light from the person to be examined. The short wavelength-side NIR light component of the incident light is an infrared light component having a wavelength in a wavelength region on a shorter wavelength side than the short wavelength infrared region (e.g., 750 nm or more and less than 900 nm) in the light from the person to be examined. The VIS light component of the incident light is a light component having a wavelength in a visible light region (e.g., 400 nm or more and less than 750 nm) in the light from the person to be examined. The dichroic prism 223 splits the observation light into the SWIR light component in one direction orthogonal to the incident direction of the observation light, and splits the observation light into the VIS light component in the other direction orthogonal to the incident direction of the observation light. The dichroic prism 223 transmits (straightly advances) the short wavelength-side NIR light component of the observation light.

The first imaging unit 224 is an image sensor that exposes incident light and outputs an image signal obtained by photoelectrically converting the exposed light, and is an image sensor that is sensitive to light in a visible region and outputs a VIS light component image (VIS image) signal. On the image plane of the first imaging unit 224, color filters of three primary colors of red (R), green (G), and blue (B), or cyan (C), magenta (M), and yellow (Y) are arranged in a Bayer array or a honeycomb array.

The second imaging unit 225 is an image sensor that exposes incident light and outputs an image signal obtained by photoelectrically converting the exposed light, and is a monochrome image sensor that is sensitive to light in a wavelength region on a shorter wavelength side than the short wavelength infrared region of light in the near-infrared region. The second imaging unit 225 outputs a short wavelength-side NIR light component image (short wavelength-side NIR image) signal.

The third imaging unit 226 is an image sensor that exposes incident light and outputs an image signal obtained by photoelectrically converting the exposed light, and is a monochrome image sensor that is sensitive to light in the short wavelength infrared region of light in the near-infrared region. The third imaging unit 226 outputs an SWIR light component image (SWIR image) signal.

Note that, in the present embodiment, the positions of the first imaging unit 224, the second imaging unit 225, and the third imaging unit 226 on optical paths are adjusted such that a focus position becomes a position (image plane) of each sensor according to the wavelength of the light component (VIS light component, short wavelength-side NIR light component, or SWIR light component) received by each of the sensors.

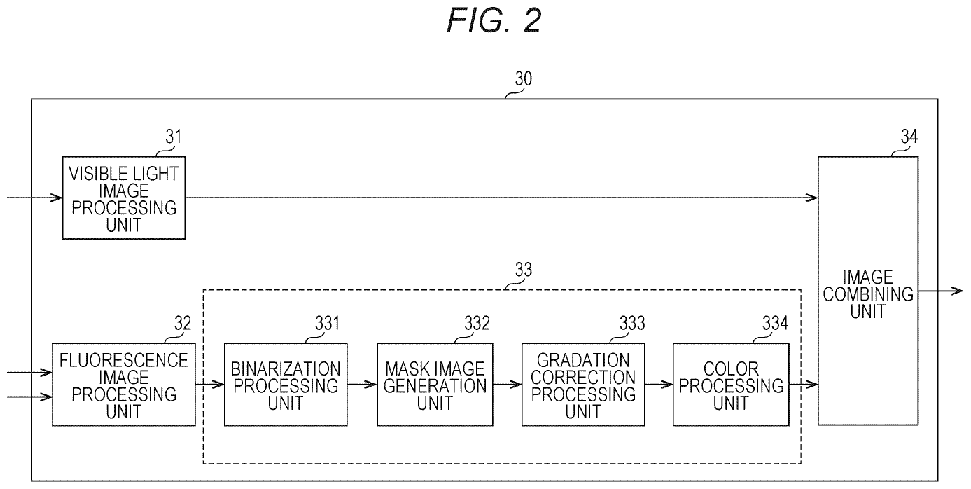

The image processing unit 30 is an image processing device that performs image processing to be described below on the image signal input from the imaging unit 22 to generate an image of the observation target. FIG. 2 is a block diagram illustrating a functional configuration of the image processing unit 30. As illustrated in FIG. 2, the image processing unit 30 includes a visible light image processing unit 31 that performs predetermined image processing suitable for a visible light image in response to the input visible light image signal and outputs the visible light image, a fluorescence image processing unit 32 that performs predetermined image processing suitable for a fluorescence image in response to the input fluorescence image signal and outputs the fluorescence image, an image correction unit 33 that generates a mask image by extracting a contour component from an SWIR image, and corrects a blur of short wavelength-side NIR image using the mask image, and an image combining unit 34 that combines the fluorescence image signal output from the image correction unit 33 with the visible light image signal output from the visible light image processing unit 31.

In addition, the image correction unit 33 includes a binarization processing unit 331 that converts an SWIR image signal into two values of black and white, a mask image generation unit 332 that creates a mask image by tracing an outer periphery of a binarized image and filling the inside of the traced outer periphery, a gradation correction processing unit 333 that sets a signal value to 0 in a bright image for a region having no signal in the mask image, and a color processing unit 334 that converts a brightness signal into a color signal.

The monitor 40 is a display device that displays the image input from the image processing unit 30, such as a liquid crystal display (LCD).

[Formation of Image]

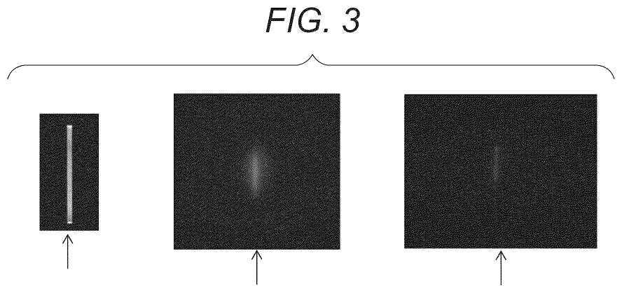

First, an image according to the present embodiment will be described. FIG. 3 is a diagram illustrating photographs representing a short wavelength-side NIR image obtained by directly imaging a test specimen included in an observation target example when is irradiated with excitation light, a short wavelength-side NIR image of the observation target example, and an SWIR image of the observation target example, respectively, in the first embodiment of the present invention. FIG. 3 illustrates, in order from the left, a short wavelength-side NIR image obtained by directly imaging the test specimen irradiated with excitation light, a short wavelength-side NIR image obtained by imaging the observation target example irradiated with excitation light through loin ham to be described below, and an SWIR image obtained by imaging the observation target example irradiated with excitation light through loin ham to be described below. In addition, an arrow in FIG. 3 indicates a position of an image of the test specimen.

The test specimen is a glass capillary tube sealing an ICG solution. The test specimen corresponds to a tissue in which ICG is accumulated in a living body, and is equivalent to the above-described “specific region”. The short wavelength-side NIR image directly captured in FIG. 3 is an image when the test specimen is irradiated with excitation light. Irradiation with excitation light having a wavelength of 808 nm maximizes excitation efficiency of ICG, and emits near-infrared fluorescence having a maximum fluorescence wavelength of about 835 nm. The fluorescence of the test specimen is clear as shown in FIG. 3.

The observation target example is a test specimen on which two slices of loin ham each having a thickness of 1.5 mm are piled up. The loin ham corresponds to a biological tissue interposed between a tissue in which ICG is accumulated in a living body and a probe. The observation target example imitates a biological tissue in which ICG is fixed in a blood vessel.

The short wavelength-side NIR image of the observation target example is an image captured from the biological tissue example side by irradiating the observation target example with excitation light from the biological tissue example side. The short wavelength-side NIR image is an image obtained by imaging light having a wavelength of 830 to 900 nm with a bandpass filter disposed in front of a VIS-SWIR compatible lens to be described below, which is attached to a VIS-SWIR camera to be described below. As illustrated in FIG. 3, the short wavelength-side NIR image of the observation target example is an image that is sufficiently bright but has a low resolution (a high-brightness and low-resolution image) because a part of the fluorescence of ICG is scattered by the biological tissue example.

The SWIR image is an image captured with a 900 nm long pass filter disposed in front of a VIS-SWIR compatible lens to be described below, which is attached to a VIS-SWIR camera. As illustrated in FIG. 3, the SWIR image has a high resolution but has a low brightness (a low-brightness and high-resolution image) because it is an image of fluorescence of ICG having a wavelength at which the fluorescence of ICG is less likely to be scattered by the biological tissue example.

In the present embodiment, the SWIR light may be light having a wavelength in a partial range of 900 to 1600 nm, light having a wavelength in the entire range of 900 to 1600 nm, or light having a wavelength in a range wider than and including the entire range of 900 to 1600 nm. Similarly, in the present embodiment, the short wavelength-side NIR light may be light having a wavelength in a partial range of 750 nm or more and less than 900 nm, light having a wavelength in the entire range of 750 nm or more and less than 900 nm, or light having a wavelength in a range wider than and including the entire range of 750 nm or more and less than 900 nm. In addition, in the present embodiment, the VIS light may be light having a wavelength in a partial range of 400 nm or more and less than 750 nm, light having a wavelength in the entire range of 400 nm or more and less than 750 nm, or light having a wavelength in a range wider than and including the entire range of 400 nm or more and less than 750 nm.

In addition, in the present embodiment, the wavelength region of the short wavelength-side NIR light and the wavelength region of the SWIR light may partially overlap with each other. In a case where the short wavelength-side NIR light and the SWIR light are set in overlapping wavelength regions, light on the shorter wavelength side or light set in a wavelength region on the shorter wavelength side is the short wavelength-side NIR light, and light on the longer wavelength side or light set in a wavelength region on the longer wavelength side is the SWIR light. The wavelength region on the shorter wavelength side is one wavelength region of which a lower limit value and an upper limit value are lower than those of the other wavelength region, of the two wavelength regions partially overlapping each other. The wavelength region on the longer wavelength side is one wavelength region of which a lower limit value and an upper limit value are higher than those of the other wavelength region, of the two wavelength regions partially overlapping each other.

[Imaging]

Next, image formation in the image forming apparatus 1 will be described.

The light source 10 emits visible light and excitation light from the distal end portion of the hard insertion part 21 to irradiate the observation target of the person to be examined. By doing so, visible light and excitation light reflected from a subject to be examined and fluorescence emitted when ICG is excited by the excitation light are incident on the first optical system 211. The first optical system 211 guides the excitation light, the visible light, and the fluorescence incident thereon to the second optical system 221 provided in the imaging unit 22.

The second optical system 221 emits the excitation light, the visible light, and the fluorescence incident from the first optical system 211 to the excitation light cut filter 222. The excitation light cut filter 222 emits light (visible light and fluorescence) obtained by attenuating the excitation light to the dichroic prism 223. The dichroic prism 223 separates a VIS light component emitted from the excitation light cut filter 222, a short wavelength-side NIR component including the fluorescence, and an SWIR light component also including the fluorescence into an optical path to the first imaging unit 224, an optical path to the second imaging unit 225, and an optical path to the third imaging unit 226.

The first imaging unit 224 exposes the VIS light emitted from the dichroic prism 223, and outputs an image signal corresponding to the VIS light to the image processing unit 30. The second imaging unit 225 exposes the short wavelength-side NIR light emitted from the dichroic prism 223, and outputs an image signal corresponding to the short wavelength-side NIR light to the image processing unit 30. The third imaging unit 226 exposes the SWIR light emitted from the dichroic prism 223, and outputs an image signal corresponding to the SWIR light to the image processing unit 30. As described above, each of the first imaging unit 224, the second imaging unit 225, and the third imaging unit 226 included in the imaging unit 22 outputs an image signal obtained by capturing an image to the image processing unit 30.

[Formation of Composite Image]

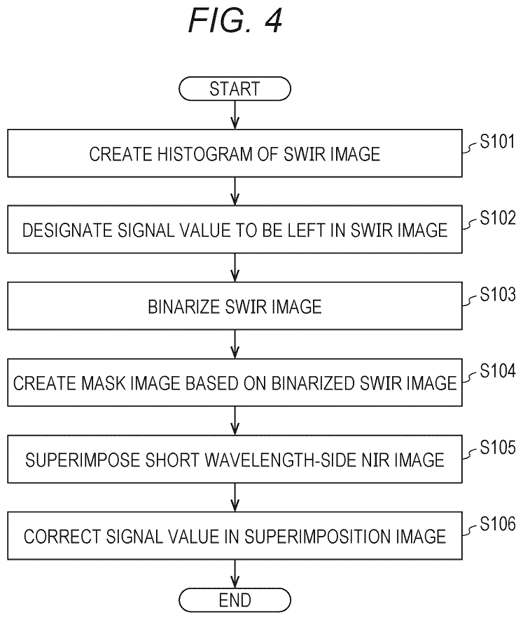

Next, image formation using the above-described images in the present embodiment will be described. FIG. 4 is a flowchart illustrating an example of an image forming process according to the first embodiment of the present invention.

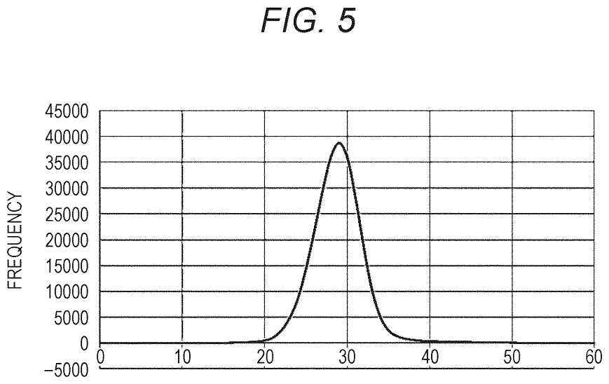

In step S101, the fluorescence image processing unit 32 creates a histogram of the SWIR image. FIG. 5 is a diagram illustrating an example of a histogram of an SWIR image according to the first embodiment of the present invention.

Specifically, the fluorescence image processing unit 32 generates an image based on an image signal corresponding to the short wavelength-side NIR light input from the second imaging unit 225 included in the imaging unit 22, that is, a short wavelength-side NIR image having a high brightness but a low resolution (blurring resulting from scattering of light by the living body is significant). The short wavelength-side NIR image is an image having an image density corresponding to the short wavelength-side NIR light, and corresponds to a second image including a specific region. Also, the fluorescence image processing unit 32 generates an image based on an image signal corresponding to the SWIR light input from the third imaging unit 226 included in the imaging unit 22, that is, an SWIR image having a low brightness but a high resolution (blurring resulting from scattering of light by the living body is significant is slight). The SWIR image is a low-brightness and high-resolution image. Therefore, the histogram of the SWIR image generated by the third imaging unit 226 shows a normal distribution-like shape with a peak at a portion of a pixel value corresponding to the test specimen.

In step S102, the binarization processing unit 331 designates a signal value to be left in the SWIR image. For example, referring to the histogram, the binarization processing unit 331 determines the number of pixels larger than the number of pixel values having a large increase or decrease in change amount in the histogram as the signal value to be left. For example, assuming that the lower limit of the signal value corresponds to a top 0.5% signal, the binarization processing unit 331 obtains a signal value corresponding to this signal from the histogram of FIG. 5, and sets this value as a threshold value.

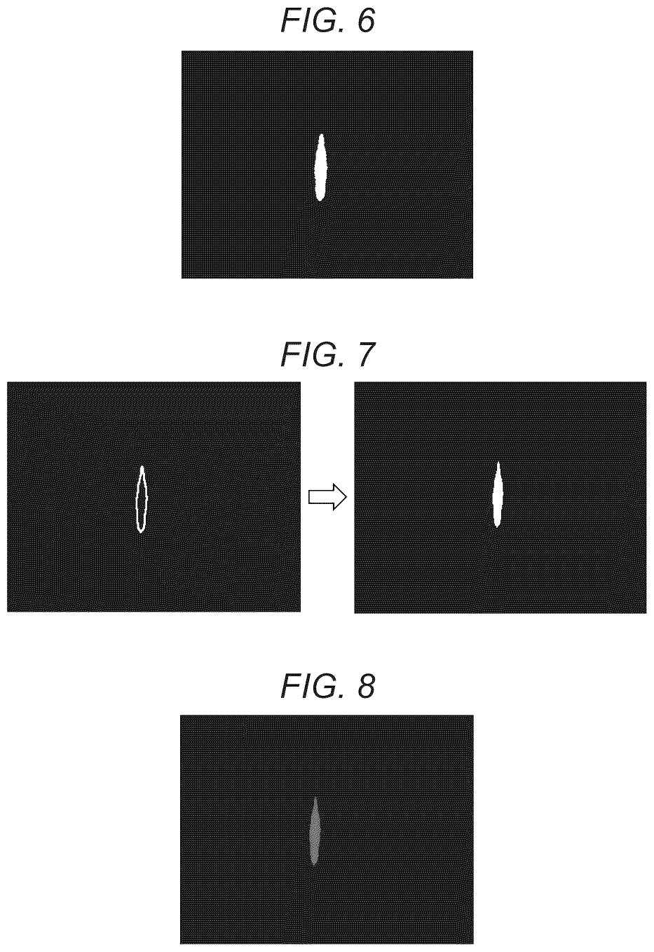

In step S103, the binarization processing unit 331 binarizes the SWIR image. FIG. 6 is a diagram illustrating an example of a binarized SWIR image according to the first embodiment of the present invention. The binarization processing unit 331 creates an image by binarizing the SWIR image using the threshold value set by the binarization processing unit 331. The binarized image corresponds to a first image indicating a boundary of the above-described specific region. As compared with the original image on the left side of FIG. 3, it can be seen that the main structural portion of the original image is left in the SWIR image.

In step S104, the mask image generation unit 332 creates a mask image based on the binarized SWIR image. FIG. 7 is a diagram illustrating an example of a contour of a binarized SWIR image and an example of a mask image formed based on the contour according to the first embodiment of the present invention. In order to obtain a portion corresponding to the signal of the binarized image of FIG. 6, the mask image generation unit 332 traces the contour of the image of FIG. 6 (see the left diagram of FIG. 7) and fills the inside of the contour to create a mask image illustrated in the right diagram of FIG. 7. A region to be left outside the contour in the mask image can be determined by what percent of the above-described histogram to be left from the top.

In step S105, the gradation correction processing unit 333 creates a superimposition image as a composite image by superimposing the mask image on the short wavelength-side NIR image. FIG. 8 is a diagram illustrating an example of a composite image obtained by superimposing a mask image on a short wavelength-side NIR image according to the first embodiment of the present invention. The gradation correction processing unit 333 superimposes the mask image generated by the mask image generation unit 332 on the short wavelength-side NIR image. In the superimposition image (composite image), the inside of the contour determined from the first image having a low brightness and a high resolution is constituted by a portion of the second image having a high brightness and a low resolution. Therefore, both the high resolution information of the first image and the high brightness information of the second image are provided.

The image processing unit 30 corrects the superimposition image if necessary. That is, in step S106, the image processing unit 30 appropriately corrects a signal value in the superimposition image. For example, the gradation correction processing unit 333 sets the signal value to 0 for a region having no signal in the mask image. By making such a correction, the image processing unit 30 can correct an influence of scattering of observation light by a living body and obtain a composite image having a high contrast, as compared with the conventional device.

In this way, the image correction unit 33 creates a mask image by extracting a contour component from the SWIR image generated by the third imaging unit 226, and filling the inside of the contour. Then, the image correction unit 33 superimposes the mask image on the short wavelength-side NIR image generated by the second imaging unit 225, and sets the signal value to 0 for a region having no signal in the mask image. In this way, the image correction unit 33 combines a high-brightness fluorescent image in which the influence of scattering of light by the living body is corrected, as compared with the conventional device.

The fluorescence image may be directly combined with the VIS image as it is, but this may deteriorate the visibility of the contour of the fluorescence image because the fluorescence image is a monochrome signal. Therefore, in step S106, the color processing unit 334 appropriately processes the color of the fluorescence image. The color processing unit 334 sets the color of the image inside the contour on the basis of various criteria. For example, in order to improve visibility, the color processing unit 334 may set the color of the image inside the contour to a color that conforms to the color of the actual biological tissue or does not exist in the biological tissue, or may set the color of the image inside the contour to a color corresponding to an intensity of the fluorescence of ICG to be multi-valued.

Then, the image combining unit 34 combines the fluorescence image subjected to appropriate color processing with the VIS image acquired by the visible light image processing unit 31 to generate a composite image. For example, the image combining unit 34 of the image processing unit 30 combines the VIS image generated by the visible light image processing unit 31 and the fluorescence image generated by the image correction unit 33 at a predetermined ratio.

The image processing unit 30 outputs data of the composite image combined by the image combining unit 34 to the monitor 40. The monitor 40 displays the composite image. The user achieves the purpose of observing the observation target by visually recognizing the composite image displayed on the monitor 40.

With such a configuration, the image forming apparatus 1 excites ICG administered to a person to be examined with excitation light, and presents an image of an observation target based on fluorescence emitted by the excited ICG to a person who performs the examination.

### Summary of First Embodiment

As is clear from the above description, the image forming apparatus 1 according to the present embodiment includes: an excitation light source (light source 10) that irradiates an observation target with excitation light; an imaging part (20) that receives first infrared light (SWIR light) and second infrared light (short wavelength-side NIR light) split from light from the observation target irradiated with the excitation light, the first infrared light including light having a wavelength in a short wavelength infrared region, and the second infrared light including light having a wavelength in a wavelength region on a shorter wavelength side than the short wavelength infrared region; and an image processing unit (30) that generates a composite image by combining a first image (SWIR image) and a second image (NIR image), the first image indicating a boundary of a specific region corresponding to the first infrared light received by the imaging part, and the second image including a specific region having an image density corresponding to the second infrared light received by the imaging part. Therefore, in an ICG examination, the image forming apparatus 1 can acquire a composite image having both a feature of a high-brightness image based on SWIR and a feature of a high-resolution image based on NIR.

The image processing unit may include an infrared image combining unit (fluorescent image processing unit 32) that generates the first image indicating a contour of an image of the specific region by binarizing an amount of the first infrared light received by the imaging part, and combines the first image and the second image. This configuration is more effective from the viewpoint that the high resolution feature of the SWIR image is definitely and easily reflected in the composite image.

The imaging part may include a splitting unit (dichroic prism 223) that splits the light from the observation target irradiated with the excitation light into a component of the first infrared light and a component of the second infrared light. This configuration is more effective from the viewpoint that a precise composite image having substantially no positional information deviation is easily created because each of data of the short wavelength-side NIR image and the SWIR image to be provided for use in the processing of the composite image is created based on the same original image.

The splitting unit may further split the light from the observation target irradiated with the excitation light into light (VIS light) having a visible light wavelength. This configuration is more effective from the viewpoint that the information of the VIS image can be reflected in the composite image, making it easy to check an image of the observation target and observe the observation target.

The image processing unit may further include a visible light image processing unit (31) that generates a visible light image corresponding to the light having a visible light wavelength received by the imaging part, and the visible light image may be added to the image obtained by combining the first image and the second image at a specific ratio to generate a composite image. With this configuration, it is possible to incorporate visible light information into the composite image (for example, setting a region outside a contour based on the second image as the visible light image). Therefore, this configuration is more effective from the viewpoint that an examination result based on the created composite image is presented in a more comprehensible manner.

The imaging part may include a filter (excitation light cut filter 222) that cuts the excitation light from the light from the observation target irradiated with the excitation light. This configuration is more effective from the viewpoint that an influence of the excitation light on image combination is reduced.

The image processing unit may generate a composite image in which a brightness signal of a region outside the specific region in the first image is corrected to zero. This configuration is more effective from the viewpoint that it is possible to definitely show only a superimposition image obtained by superimposing the first image and the second image on each other, thereby presenting an examination result based on the created composite image in an easy and definite manner.

Other embodiments of the present invention will be described below. In the following description of each of the embodiments, for convenience of description, members having the same functions as those described in the above-described embodiment will be denoted by the same reference signs, and the description thereof will not be repeated.

### Second Embodiment

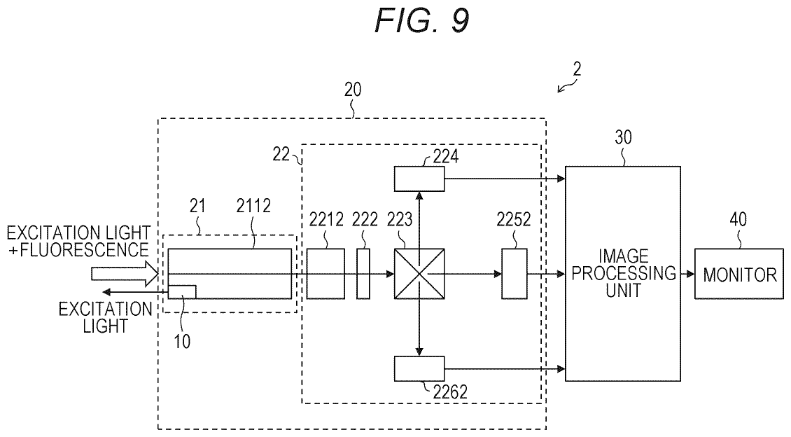

FIG. 9 is a diagram schematically illustrating a functional configuration of an image forming apparatus according to a second embodiment of the present invention. As illustrated in FIG. 9, an image forming apparatus 2 has the same configuration as the image forming apparatus 1 according to the first embodiment described above, except that the configurations of the hard insertion part 21 and the imaging unit 22 are partially different.

The hard insertion part 21 includes a first optical system 2112 instead of the first optical system 211, and the imaging unit 22 includes a second optical system 2212 instead of the second optical system 221. In both an objective lens of the second optical system 2212 and an image forming lens of the second optical system 2212, a focus shift is corrected for light in a wavelength region (e.g., 400 to 1600 nm) of VIS light and NIR light.

The VIS light and the NIR light are transmitted through the imaging part 20. For this reason, a wavelength-dependent focus shift may occur due to a wavelength-dependent difference in refractive index in the lens. Therefore, from the viewpoint that such a focus shift is prevented, in the present embodiment, an optical system having high image forming performance in which an aberration is well corrected in a wide wavelength region from the visible light region to the short wavelength infrared region is required. In the present embodiment, focus shifts are corrected in the first optical system 2112 and the second optical system 2212, from the viewpoint that a wavelength-dependent focus shift(FS) is suppressed to enhance coupling performance.

More specifically, each of the first optical system 2112 and the second optical system 2212 is optically designed to reduce a deviation of a back focus (BF) position in the VIS region, the short wavelength-side NIR region, and the SWIR region. The focus position in the present embodiment is a focus position on a paraxial ray (a ray passing through a height very close to an optical axis). Note that a “back focus” (BF) refers to a distance from a surface of the optical system closest to the image to the focus position, and a value of the focus position does not change even when an F number of the lens changes.

In the present embodiment, from the viewpoint that an aberration of an image detected by each sensor is well corrected, the first optical system preferably satisfies the following formula (1), and the second optical system preferably satisfies the following formula (2). In the following formulas, “BF 550 nm” represents a back focus in the entire optical system of 550 nm, “BF 850 nm” represents a back focus in the entire optical system of 850 nm, and “BF 1600 nm” represents a back focus in the entire optical system of 1600 nm.

|BF_550 nm−BF_850 nm|<0.03 (1)

|BF_550 nm−BF_1600 nm|<0.05 (2)

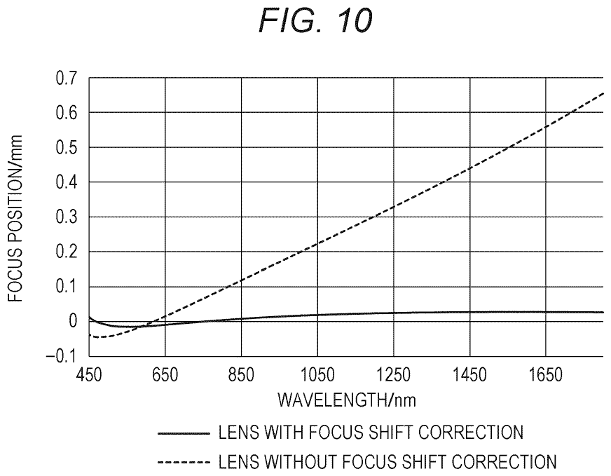

In the present embodiment, back focus values in entire optical systems when various products are used are shown in Table 1. In addition, FIG. 10 illustrates a relationship of a focus position with each wavelength of each of a lens with focus shift correction as product C and a lens without focus shift correction in Table 1. In FIG. 10, a solid line indicates a focus position of a lens with focus shift correction, and a broken line indicates a focus position of a lens without focus shift correction. It can be seen from FIG. 10 that a deviation of a focus position is corrected particularly on the long wavelength side by using the first optical system and the second optical system in which focus shifts are corrected.

TABLE 1

| | | | | | Lens |
|---|---|---|---|---|---|
| | Lens with FS correction | | | | without FS |
| Product | A | B | C | D | correction |
| Focal length/mm | 35 | 12 | 16 | 25 | 16 |
| F number | 1.5 | 1.6 | 1.6 | 1.6 | 1.8 |
| Focus position at | 0.015 | 0.014 | −0.015 | 0.017 | −0.029 |
| 550 nm [mm] | | | | | |
| Focus position at | 0.024 | 0.011 | 0.008 | 0.032 | 0.119 |
| 850 nm [mm] | | | | | |
| Focus position at | 0.030 | 0.039 | 0.028 | 0.039 | 0.528 |
| 1600 nm [mm] | | | | | |
| BF 550 nm-850 nm | 0.009 | 0.003 | 0.023 | 0.015 | 0.148 |
| [mm] | | | | | |
| BF 550 nm-1600 | 0.015 | 0.025 | 0.043 | 0.022 | 0.557 |
| nm [mm] | | | | | |

The imaging unit 22 includes a second imaging unit 2252 instead of the second imaging unit 225, and includes a third imaging unit 2262 instead of the third imaging unit 226. The second imaging unit 2252 and the third imaging unit 2262 include VIS-SWIR sensors. The VIS-SWIR sensor is an element that enables imaging of VIS light to SWIR light. More specifically, the second imaging unit 2252 and the third imaging unit 2262 can have the following configurations.

Camera (image sensor): camera BH-71IGA (manufactured by BITRAN) on which a VIS-SWIR sensor (sensitivity to 400 to 1700 nm) is mounted

Lens: VIS-SWIR compatible lens (transmission band of 400 to 1700 nm with focus shift correction)

In the present embodiment, both the second imaging unit 2252 and the third imaging unit 2262 can detect images of light from VIS light to SWIR light. In both the first optical system 2112 and the second optical system 2212, focus shifts are corrected. In the present embodiment, the imaging devices can be made common, and it is not necessary to adjust a position of each of the imaging devices according to a focus shift. Therefore, the present embodiment is more suitable, from the viewpoint that the optical design of the imaging part 20 is simplified in addition to the features of the first embodiment described above.

### Third Embodiment

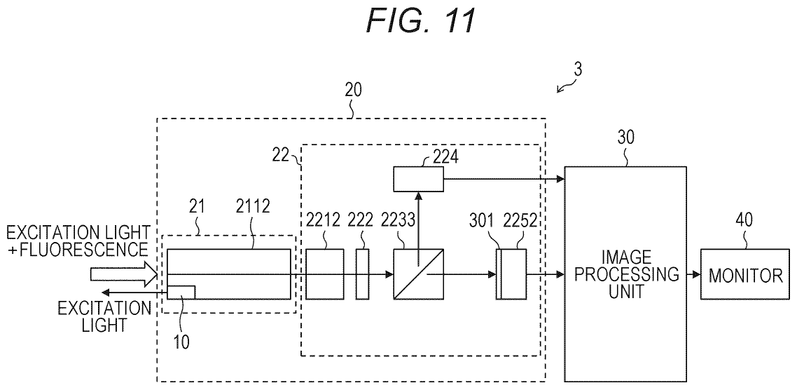

FIG. 11 is a diagram schematically illustrating a functional configuration of an image forming apparatus according to a third embodiment of the present invention. As illustrated in FIG. 11, an image forming apparatus 3 has the same configuration as the image forming apparatus 2 according to the second embodiment described above, except that the configuration of the imaging unit 22 is partially different.

The imaging unit 22 includes a dichroic prism 2233 instead of the dichroic prism 223. Furthermore, the imaging unit 22 does not include the third imaging unit 2262, but further includes an NIR-SWIR filter 301 corresponding to the second imaging unit 2252 instead.

The dichroic prism 2233 is a beam splitter that splits incident light into a VIS light component in a different direction. The dichroic prism 2233 splits the observation light into the VIS light component in one direction orthogonal to the incident direction of the observation light, and transmits (straightly advances) the short wavelength-side NIR light component and the SWIR light component of the observation light.

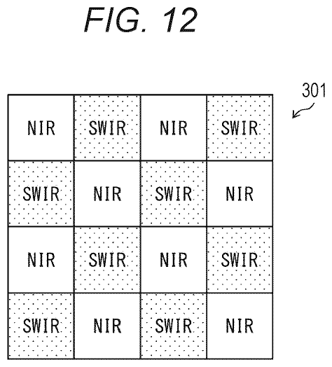

FIG. 12 is a diagram schematically illustrating a configuration of an NIR-SWIR filter according to the third embodiment of the present invention. As illustrated in FIG. 12, an NIR-SWIR filter 301 includes NIR filters that transmit substantially only short wavelength-side NIR light and SWIR filters that transmit substantially only SWIR light. In the NIR-SWIR filter 301, the NIR filters and the SWIR filters are arranged, for example, in a checkered pattern as illustrated.

In the present embodiment, one second imaging unit 2252 captures both a short wavelength-side NIR image and an SWIR image. Therefore, the present embodiment is suitable from the viewpoint that the configuration of the imaging part 20 is further simplified in addition to the features of the second embodiment described above.

### Fourth Embodiment

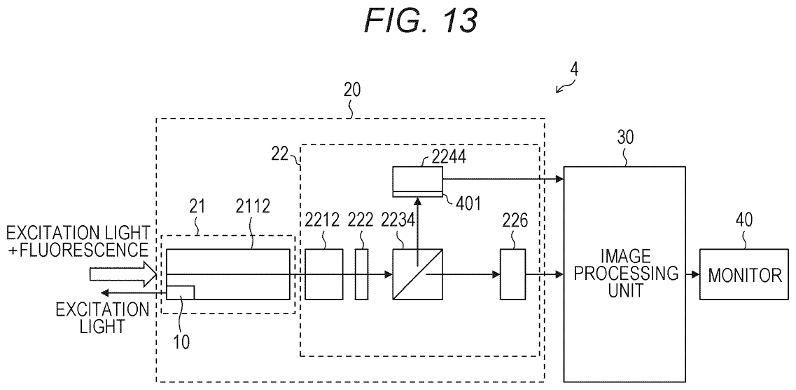

FIG. 13 is a diagram schematically illustrating a functional configuration of an image forming apparatus according to a fourth embodiment of the present invention. As illustrated in FIG. 13, an image forming apparatus 4 has the same configuration as the image forming apparatus 3 according to the third embodiment described above, except that the configuration of the imaging unit 22 is partially different.

The imaging unit 22 includes a dichroic prism 2234 instead of the dichroic prism 2233. Furthermore, the imaging unit 22 includes a first imaging unit 2244 instead of the first imaging unit 224, and further includes a VIS-NIR filter 401 corresponding to the first imaging unit 2244.

The dichroic prism 2234 is a beam splitter that splits incident light into a VIS light component and a short wavelength-side NIR light component in a different direction from a SWIR light component. The dichroic prism 2234 splits the observation light into the VIS light component and the short wavelength-side NIR light component in one direction orthogonal to the incident direction of the observation light, and transmits (straightly advances) the SWIR light component of the observation light.

The first imaging unit 2244 includes a VIS-NIR sensor. The VIS-NIR sensor is an element that enables imaging of VIS light to short wavelength-side NIR light.

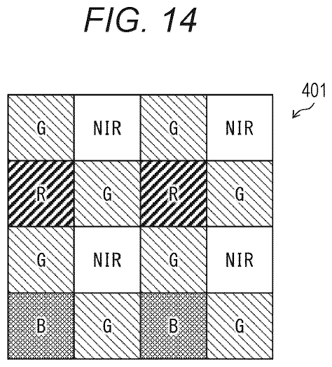

FIG. 14 is a diagram schematically illustrating a configuration of a VIS-NIR filter according to the fourth embodiment of the present invention. As illustrated in FIG. 14, for example, a VIS-NIR filter 401 includes RGB color filters and NIR filters arranged in a Bayer array, the RGB color filters transmitting VIS light, and the NIR filters transmitting substantially only short wavelength-side NIR light.

In the present embodiment, one first imaging unit 2244 captures both a VIS image and a short wavelength-side NIR image. Therefore, the present embodiment is suitable from the viewpoint that the configuration of the imaging part 20 is further simplified, similarly to the features of the third embodiment described above.

### Fifth Embodiment

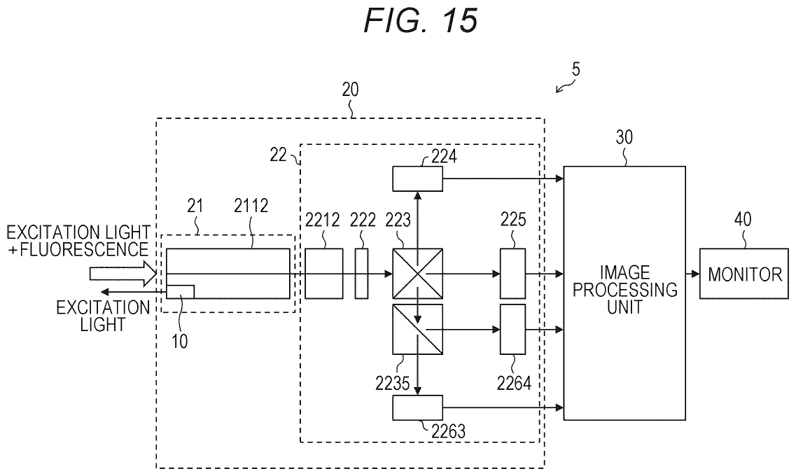

FIG. 15 is a diagram schematically illustrating a functional configuration of an image forming apparatus according to a fifth embodiment of the present invention. As illustrated in FIG. 15, an image forming apparatus 5 has the same configuration as the image forming apparatus 1 according to the first embodiment described above, except that the configurations of the hard insertion part 21 and the imaging unit 22 are partially different.

The hard insertion part 21 includes a first optical system 2112 instead of the first optical system 211, and the imaging unit 22 includes a second optical system 2212 instead of the second optical system 221. Furthermore, the imaging unit 22 includes a dichroic prism 2235 and third imaging units 2263 and 2264 instead of the third imaging unit 226.

The dichroic prism 2235 is a beam splitter that splits incident light into SWIR light components in different directions based on wavelengths thereof. The dichroic prism 2235 separates a long wavelength-side component (e.g., 1000 to 1600 nm) of the SWIR light components in one direction orthogonal to the light incident direction, and transmits (straightly advances) a short wavelength-side component (e.g., 900 nm or more and less than 1000 nm) of the SWIR light components.

Each of the third imaging units 2263 and 2264 includes an SWIR sensor. The third imaging unit 2263 exposes a light component of 900 nm or more and less than 1000 nm of the SWIR light emitted from the dichroic prism 2235, and outputs an image signal corresponding to the light component having the wavelength of 900 nm or more and less than 1000 nm to the image processing unit 30. The third imaging unit 2264 exposes a light component of 1000 to 1600 nm of the SWIR light emitted from the dichroic prism 2235, and outputs an image signal corresponding to the light component having the wavelength of 1000 to 1600 nm to the image processing unit 30.

Then, the fluorescence image processing unit 32 generates SWIR images in the respective wavelength regions, based on the image signals corresponding to the SWIR light components input from the third imaging unit 2263 and the third imaging unit 2264 provided in the imaging unit 22.

In the present embodiment, the third imaging unit 2263 captures an SWIR image of 900 nm or more and less than 1000 nm, and the third imaging unit 2264 captures an SWIR image of 1000 to 1600 nm. The fluorescence intensity of ICG has a chevron peak characteristic in which the fluorescence intensity gradually decreases from about 835 nm to 1600 nm. Therefore, a bright and high-resolution SWIR image can be obtained from the SWIR light component of 900 nm or more and less than 1000 nm. In addition, from the SWIR light component of 1000 to 1600 nm, an SWIR image having a lower brightness but a higher resolution can be obtained as compared with that from the SWIR light component of 900 nm or more and less than 1000 nm. Therefore, for example, when the ICG administered to the observation target is located at a deep position in the body, brightness can be emphasized by using an SWIR image of 900 nm or more and less than 1000 nm, and when the ICG administered to the observation target is located at a shallow position in the body, resolution can be emphasized by using an SWIR image of 1000 to 1600 nm.

As described above, the present embodiment is suitable from the viewpoint that an optimal SWIR image can be selected depending on a position of ICG administered to the observation target.

### Sixth Embodiment

FIG. 16 is a diagram schematically illustrating a functional configuration of an image forming apparatus according to a sixth embodiment of the present invention. As illustrated in FIG. 16, an image forming apparatus 6 has the same configuration as the image forming apparatus 1 according to the first embodiment described above, except that the configurations of the hard insertion part 21 and the imaging unit 22 are partially different.

The hard insertion part 21 includes a first optical system 2112 instead of the first optical system 211, and the imaging unit 22 includes a second optical system 2212 instead of the second optical system 221. Furthermore, the imaging unit 22 includes a dichroic prism 2236 instead of the dichroic prism 223, and includes a second imaging unit 2252 and an NIR-SWIR filter 301 instead of the second imaging unit 225.

The dichroic prism 2236 is a beam splitter that splits incident light into an NIR light component and a visible light component in different directions. The dichroic prism 2236 transmits (straightly advances) a light component of the NIR light of the observation light having a wavelength on a shorter wavelength side (e.g., 800 nm or more and less than 1000 nm). The dichroic prism 2236 splits the NIR light of the observation light into a light component having a wavelength of 1000 to 1600 nm in one direction orthogonal to the incident direction of the observation light, and splits the observation light into the VIS light component in the other direction orthogonal to the incident direction of the observation light.

In the present embodiment, from a light component of the NIR light of the incident light having a wavelength of 800 nm or more and less than 1000 nm, an image signal of a short wavelength-side NIR image according to a light component having a wavelength of 800 nm or more and less than 900 nm and an image signal of an SWIR image according to a light component having a wavelength of 900 nm or more and less than 1000 nm are obtained. In addition, in the present embodiment, an image signal of an SWIR image is further obtained from a light component having a wavelength of 1000 to 1600 nm. The detection intensity of the fluorescence of ICG varies depending on the type of detector. For example, an InGaAs-based detector has stronger sensitivity in the SWIR region than a Si-based detector. The present embodiment is suitable from the viewpoint that the SWIR light detection sensitivity is further increased in a case where a sensor having stronger sensitivity in the SWIR region is used for the second imaging unit 2252.

### Seventh Embodiment

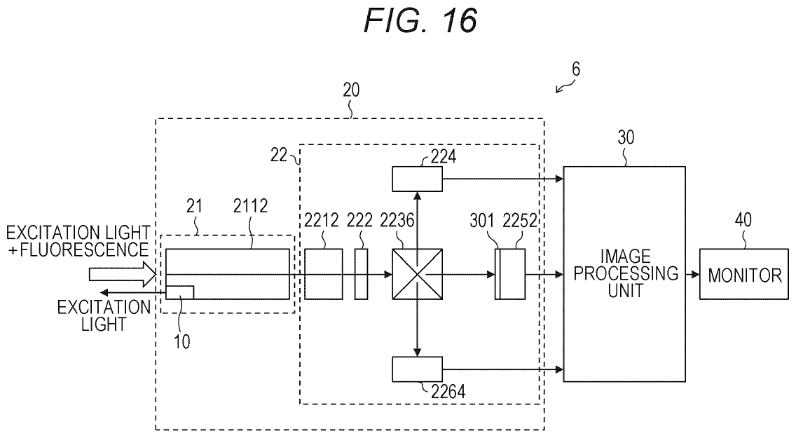

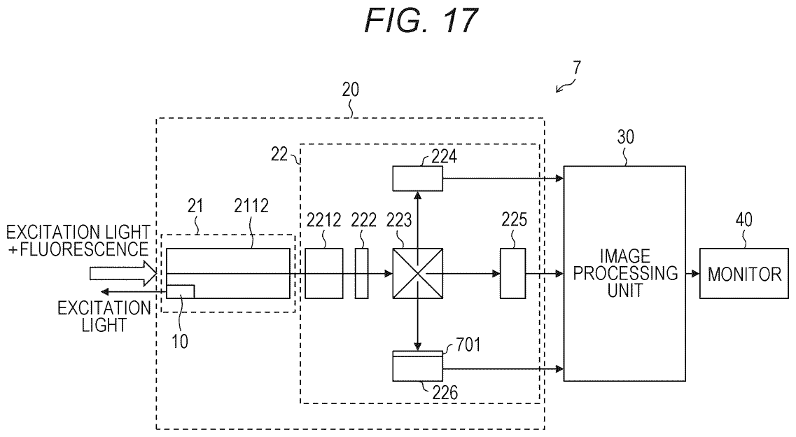

FIG. 17 is a diagram schematically illustrating a functional configuration of an image forming apparatus according to a seventh embodiment of the present invention. As illustrated in FIG. 17, an image forming apparatus 7 has the same configuration as the image forming apparatus 1 according to the first embodiment described above, except that the configurations of the hard insertion part 21 and the imaging unit 22 are partially different.

The hard insertion part 21 includes a first optical system 2112 instead of the first optical system 211, and the imaging unit 22 includes a second optical system 2212 instead of the second optical system 221. Furthermore, the imaging unit 22 further includes an SWIR-SWIR filter 701 corresponding to the third imaging unit 226.

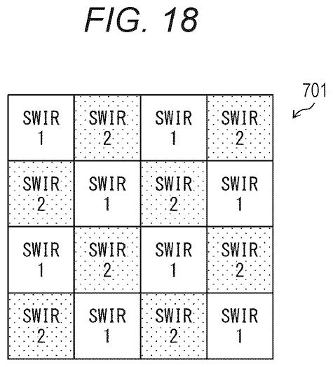

FIG. 18 is a diagram schematically illustrating a configuration of an SWIR-SWIR filter according to the seventh embodiment of the present invention. As illustrated in FIG. 18, in the SWIR-SWIR filter 701, first SWIR filters and second SWIR filters are arranged, for example, in a checkered pattern as illustrated. The first SWIR filter is a filter that substantially transmits only a light component having a wavelength on the shorter wavelength side (e.g., 900 nm or more and less than 1000 nm) of the SWIR light. The second SWIR filter is a filter that substantially transmits only a light component having a wavelength (e.g., 1000 to 1600 nm) on the longer wavelength side of the SWIR light.

Similarly to the sixth embodiment, the present embodiment is suitable from the viewpoint that the SWIR light detection sensitivity is further increased in a case where a sensor having stronger sensitivity in the SWIR region is used for the third imaging unit 226.

### Eighth Embodiment

FIG. 19 is a diagram schematically illustrating a functional configuration of an image forming apparatus according to an eighth embodiment of the present invention. As illustrated in FIG. 19, an image forming apparatus 8 includes a hard insertion part 21, an imaging unit 22, an image processing unit 80, and a monitor 40.

The hard insertion part 21 includes a light source 1010 and a first optical system 2112. The light source 1010 is a light source that intermittently irradiates an observation target with red (R) light, green (G) light, blue (B) light, and near-infrared (NIR) light in the respective wavelength bands. Specifically, the light source 1010 includes a red light source 1011 (e.g., a wavelength of 633 nm), a green light source 1012 (e.g., a wavelength of 470 nm), a blue light source 1013 (e.g., a wavelength of 525 nm), and a near-infrared light source 1014 (e.g., a wavelength of 808 nm) as an excitation light source. These light sources are connected to a control unit, which is not illustrated, and are configured to emit light at a specific timing according to a control signal from the control unit.

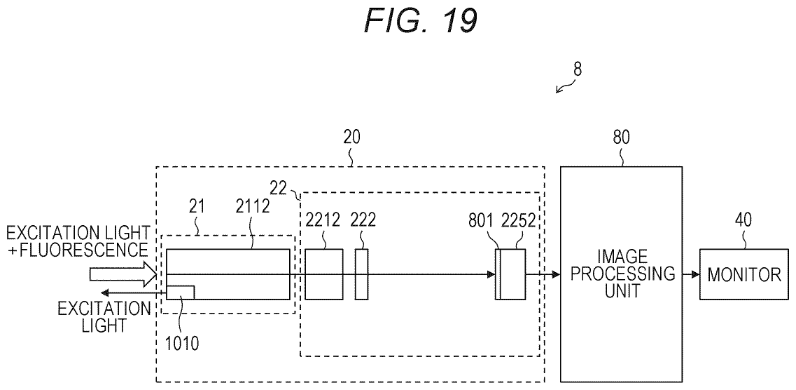

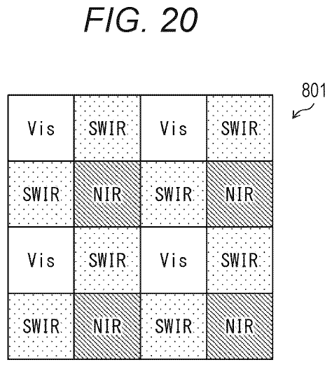

The imaging unit 22 includes a second optical system 2212, an excitation light cut filter 222, a VIS-NIR-SWIR filter 801, and a second imaging unit 2252. FIG. 20 is a diagram schematically illustrating a configuration of a VIS-NIR-SWIR filter according to the eighth embodiment of the present invention. As illustrated in FIG. 20, the VIS-NIR-SWIR filter 801 includes VIS filters that substantially transmit only VIS light, NIR filters that substantially transmit only short wavelength-side NIR light, and SWIR filters that substantially transmit only SWIR light. In the VIS-NIR-SWIR filter 801, the VIS filters, the NIR filters, and the SWIR filters are arranged in a specific pattern, for example, in a checkered pattern as illustrated.

FIG. 21 is a block diagram illustrating a functional configuration of an image processing unit of the image forming apparatus according to the eighth embodiment of the present invention. As illustrated in FIG. 21, the image processing unit 80 further includes an RGB combination processing unit 81 that receives image signals corresponding to R, G, and B input from the second imaging unit 2252, generates visible light image signals corresponding to R, G, and B components, respectively, and outputs the visible light image signals to the visible light image processing unit 31. Other than that, the image processing unit 80 has a functional configuration similar to that of the image processing unit 30 described above.

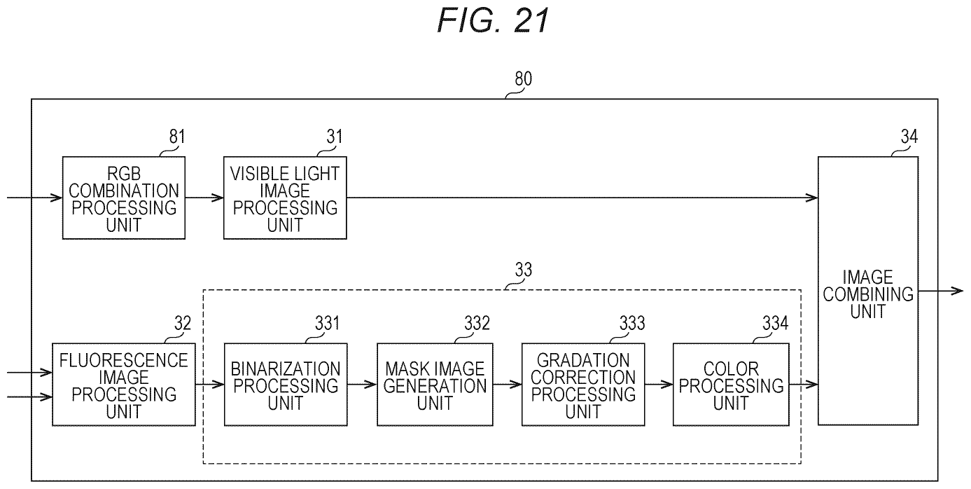

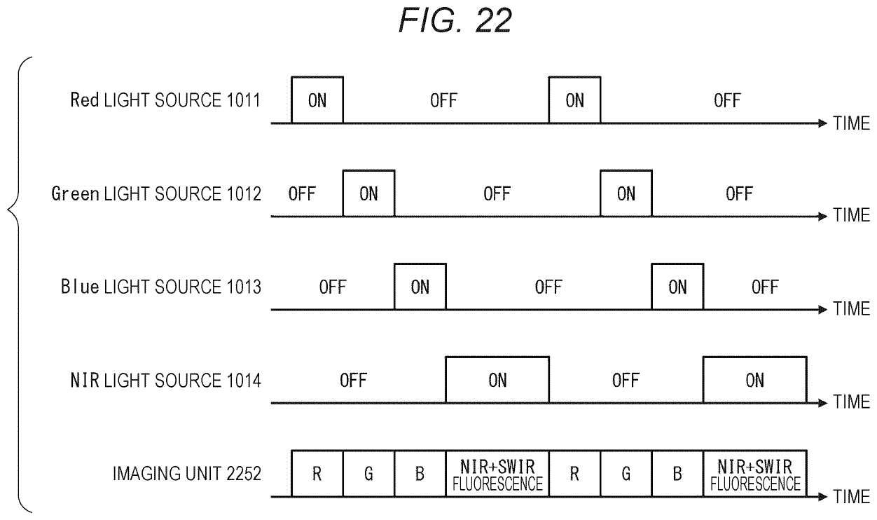

In the present embodiment, in synchronization with an imaging timing of the second imaging unit 2252, an observation target is intermittently irradiated with red (R) light, green (G) light, blue (B) light, and near-infrared (NIR) light in the respective wavelength bands, reflected light of the red (R) light, the green (G) light, and the blue (B) light from the observation target is exposed in a time division manner, and a visible light image is generated by RGB combination processing. FIG. 22 is a diagram illustrating a timing chart for explaining an example of an operation of the image forming apparatus according to the eighth embodiment of the present invention. Note that “NIR” of “NIR+SWIR fluorescence” for the imaging unit 2252 in FIG. 22 refers to “short wavelength-side NIR”.

As illustrated in FIG. 22, at the time when red light is emitted from the red light source 1011 of the light source 1010, the control unit causes the second imaging unit 2252 to output a red light image signal among light components that have passed through the VIS filter of the VIS-NIR-SWIR filter 801 and have been received by the second imaging unit 2252. Similarly, the control unit causes the second imaging unit 2252 to output a green light image signal at the time when green light is emitted from the green light source 1012, and causes the second imaging unit 2252 to output a blue light image signal at the time when blue light is emitted from the blue light source 1013. In addition, at the time when NIR light is emitted from the near-infrared light source 1014 of the light source 1010, the control unit causes the second imaging unit 2252 to output a short wavelength-side NIR light image signal that has passed through the NIR filter, and causes the second imaging unit 2252 to output an SWIR light image signal that has passed through the SWIR filter, among light components received by the second imaging unit 2252. In this way, the red light source 1011, the green light source 1012, the blue light source 1013, and the near-infrared light source 1014 of the light source 1010 are alternately activated and deactivated in a repeated manner, and the second imaging unit 2252 acquires red image signals, blue image signals, green image signals, image signals corresponding to short wavelength-side NIR light, and image signals corresponding to SWIR light for one frame.

Among these image signals, the red image signal, the blue image signal, and the green image signal are output to the RGB combination processing unit 81 of the image processing unit 80 to generate a visible light image, and an image signal corresponding to near-infrared fluorescence and an image signal corresponding to short wavelength infrared fluorescence are output to the fluorescence image processing unit 32 of the image processing unit 80.

In the present embodiment, one second imaging unit 2252 captures a VIS image, a short wavelength-side NIR image, and an SWIR image. Therefore, the present embodiment is suitable from the viewpoint that the configuration of the imaging part 20 is further simplified.

### Other Embodiments

Hereinafter, it will be described how a composite image is created according to other embodiment of the present invention.

[Creation of Composite Image by Boundary Restoration Processing]

Even a bright image (short wavelength-side NIR image) should have a definite boundary between different tissues, but the boundary between the tissues is obscure because the image is blurred. On the other hand, although a signal of a dark image (SWIR image) is small, a boundary between tissues is clear. By extracting a boundary line with a dark image (SWIR image) and superimposing the obtained boundary line on a blurred image, it is possible to reproduce a clear boundary line between tissues originally possessed by the image.

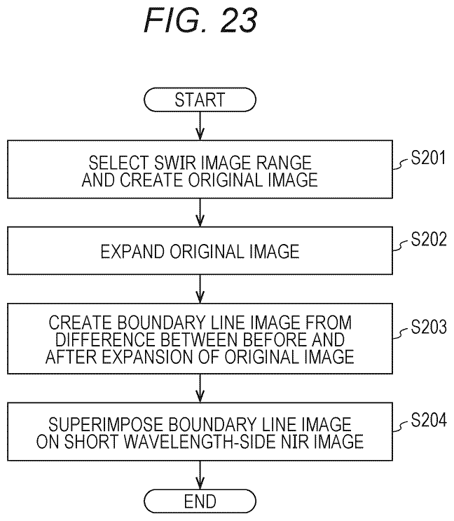

A dark image (SWIR image) with a clear boundary between tissues is used, and top-hat transformation is applied to this image. FIG. 23 is a flowchart illustrating an example of image processing for forming a composite image by top-hat transformation.

In step S201, the image processing unit 30 selects an SWIR image range and creates an original image.

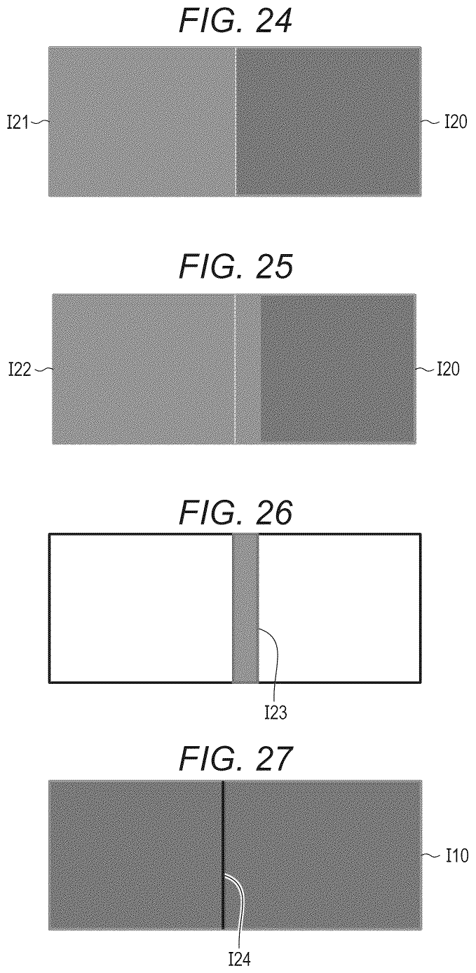

FIG. 24 is a diagram schematically illustrating an original image in the image processing for forming a composite image by top-hat transformation. The entire image can be divided into two regions according to a magnitude of a signal value of a tissue. Specifically, the image has a boundary as illustrated in FIG. 24. For example, a region of a tissue emitting fluorescence of ICG is 121. Region 120 is a region of a portion outside the tissue.

In step S202, the image processing unit 30 creates an image obtained by expanding the original image. FIG. 25 is a diagram schematically illustrating an image obtained by expanding the original image by top-hat transformation. For example, at the boundary of the region 121, the image processing unit 30 copies a pixel value of a pixel adjacent to the boundary to pixels outside the boundary and adjacent to the boundary in upward, downward, leftward, and rightward directions. As a result, a region 122 expanding from the region 121 as much as one pixel along the boundary is created. In FIG. 25, since the left side and the upper and lower sides of the region 122 in the drawing are not changed in pixel values because they are edges of the image, and only the right side of the region is enlarged.

In step S203, the image processing unit 30 creates a boundary line image from a difference between before and after the expansion of the original image. FIG. 26 is a diagram schematically illustrating an image of a boundary portion extracted in the image processing for forming a composite image by top-hat transformation. By subtracting the region 121 from the region 122, only the enlarged portion remains. In this way, a region 123 of the boundary portion corresponding to the boundary line of the tissue is created.

In step S204, the image processing unit 30 superimposes the boundary line image on a short wavelength-side NIR image. FIG. 27 is a diagram schematically illustrating a superimposition image of a boundary portion image and a second image (short wavelength-side NIR image) in the image processing for forming a composite image by top-hat transformation. An image 110 is a short wavelength-side NIR image, and a region 124 is an image obtained by multiplying a pixel value of the region 123 by a constant to accentuate the boundary portion.

As described above, by multiplying a signal of a boundary line, which is obtained by applying top-hat transformation to a dark image (SWIR image), by a constant and adding the signal of the boundary line to a bright image (short wavelength-side NIR image) in which a boundary between tissues is unclear, a clear boundary line between the tissues in the dark image (SWIR image) is reflected in the bright image (short wavelength-side NIR image) to create a composite bright image (composite image) in which the boundary between the tissues is clear.

Note that, in the image combination, the boundary portion image may be subtracted from the short wavelength-side NIR image. In this case as well, a bright composite image (composite image) with a clear boundary between tissues is created.

[Creation of Composite Image by Accentuation of Edge]

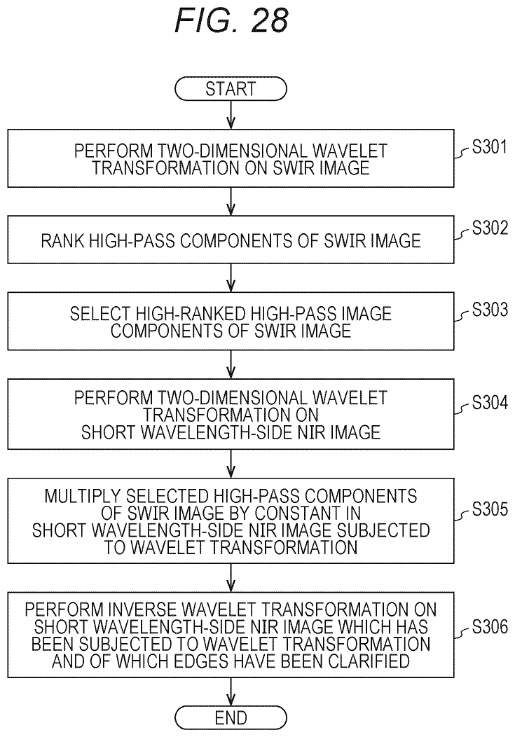

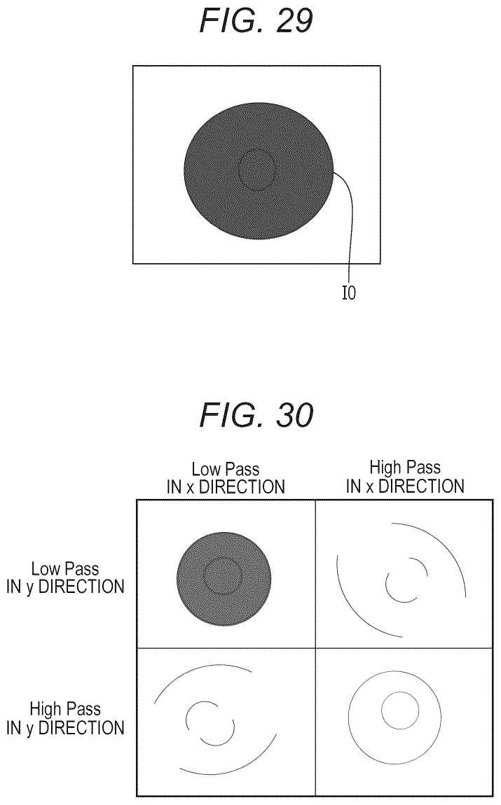

FIG. 28 is a flowchart illustrating an example of image processing for forming a composite image by wavelet transformation. FIG. 29 is a diagram schematically illustrating an original image I0 in the image processing for forming a composite image by wavelet transformation. The original image I0 is, for example, an image of a tissue emitting fluorescence of ICG.

In step S301, the image processing unit 30 performs two-dimensional wavelet transformation on an SWIR image.

In step S302, the image processing unit 30 ranks high-pass components of the SWIR image. FIG. 30 is a diagram schematically illustrating frequency component images of the original image I0 decomposed by the wavelet transformation. Edge components appears in high-pass components. When the wavelet transformation is performed on the dark image (SWIR image) as described above, the high-pass filter components corresponding to edges has a large value.

In step S303, the image processing unit 30 selects high-ranked high-pass image components of the SWIR image. The components having a large value are recorded in the high-pass filter.

In step S304, the image processing unit 30 performs two-dimensional wavelet transformation on a short wavelength-side NIR image. For example, the wavelet transformation is applied to a bright image (short wavelength-side NIR image). In the bright image (short wavelength-side NIR image) after being subjected to the wavelet transformation, since the short wavelength-side NIR image is a high-brightness and low-resolution image, components corresponding to edges become small and are buried in the components other than the edges.

In step S305, the image processing unit 30 multiplies the selected high-pass components (the components having a large value recorded in step S303) of the SWIR image by a constant in the short wavelength-side NIR image subjected to the wavelet transformation. In this way, the components in the short wavelength-side NIR image subjected to the wavelet transformation are restored using the corresponding components of the SWIR image obtained by performing the wavelet transformation. As a result, a short wavelength-side NIR image with definite edges, which is an image subjected to wavelet transformation, is created.

In step S306, the image processing unit 30 performs inverse wavelet transformation on the short wavelength-side NIR image which has been subjected to wavelet transformation and of which edges have been clarified. As a result, it is possible to obtain a composite image in which the definite edges of the dark image (SWIR image) are reflected in the bright image (short wavelength-side NIR image).

[Modifications]

The present invention is not limited to the above-described embodiments, and various modifications can be made within the scope set forth in the claims. Embodiments obtained by appropriately combining technical means disclosed in the different embodiments also fall within the technical scope of the present invention.

For example, in embodiments of the present invention, the excitation light cut filter may be a filter that transmits light having a desired wavelength or a combination of filters that transmit light having desired wavelengths. For example, the excitation light cut filter may be a combination of a band pass filter (transmission band of 830 to 900 nm) that transmits a short wavelength-side NIR component of fluorescence of ICG and a long pass filter (transmission band of 900 to 1600 nm) that transmits an SWIR component.

In addition, the excitation light cut filter may be disposed between an observation target and an image sensor, and the excitation light cut filter may be provided in the dichroic prism itself.

The functions of the image processing unit 30 in the embodiments of the present invention can be realized by a program for causing a computer to function as the image processing unit, which is a program for causing a computer to function as the respective control blocks of the image processing unit.

In this case, the processing unit includes a computer having at least one control device (e.g., a processor) and at least one storage device (e.g., a memory), as hardware for executing the program. By executing the program by the control device and the storage device, the functions described in the above embodiments are realized.

The program may be recorded in one or more non-transitory and computer-readable recording media. The processing unit may or may not be provided in the recording medium. In a case whether the processing unit is not provided in the recording medium, the program may be supplied to the processing unit via any wired or wireless transmission medium.

In addition, some or all of the functions of the control blocks can be realized by logic circuits. For example, an integrated circuit in which the logic circuits functioning as the respective control blocks are formed also falls within the scope of the present invention. In addition, for example, the respective functions of the control blocks can be realized by a quantum computer.

Each process described in each of the above-described described embodiments may be executed by artificial intelligence (AI). In this case, the AI may operate in the control device, or may operate in another device (e.g., an edge computer, a cloud server, or the like).

The image forming apparatus according to the present invention may further include a diagnosis unit that diagnoses a tissue from a created composite image. The diagnosis unit may include a determination unit that determines an image using an image determination model that has learned composite image data as teacher data. Examples of the image determination model include a neural network and a support vector machine. Examples of the neural network include a convolutional neural network (CNN), a recurrent neural network (RNN), and a fully connected neural network.

The image determination model can be trained with reference to the teacher data. The teacher data includes image data of the composite image and at least one piece of information (such as a disease at a site) related to a site in the image corresponding to the image data. The learning of the image determination model can be created by training a neural network with the sufficiently prepared teacher data (image data and information related to a site corresponding thereto), and determining a path weight for each piece of the image data. Examples of the algorithm for training the image determination model include backpropagation and ID3.

Note that the image determination model may be a model other than a model based on machine learning. For example, the image determination model may be a regression model using the above-described image data as an objective variable and information regarding whether the image data is appropriate as an explanatory variable.

In the present invention, a sufficiently bright and sufficiently clear image is obtained by supplementing clearness of an image having a sufficient image density, which is detected by light having a first wavelength, in an image having a non-sufficient image density but a high resolution, which is detected by light having a second wavelength. In the present invention, light in a specific wavelength region is used for infrared light. The present invention may be applied when there is a specific wavelength region in which the above-described brightness and clearness features of the image.

### SUMMARY

As is clear from the above description, an image forming apparatus according to a first aspect of the present invention includes: an excitation light source that irradiates an observation target with excitation light; an imaging part that receives first infrared light and second infrared light split from light from the observation target irradiated with the excitation light, the first infrared light including light having a wavelength in a short wavelength infrared region, and the second infrared light including light having a wavelength in a wavelength region on a shorter wavelength side than the short wavelength infrared region; and an image processing unit that generates a composite image by combining a first image and a second image, the first image indicating a boundary of a specific region corresponding to the first infrared light received by the imaging part, and the second image including a specific region having an image density corresponding to the second infrared light received by the imaging part. The first aspect can provide a new technology capable of acquiring an image having both a feature of a high-brightness image and a feature of a high-resolution image.

In an image forming apparatus according to a second aspect of the present invention, in the first aspect described above, the image processing unit may include an infrared image combining unit that generates the first image indicating a contour of an image of the specific region by binarizing an amount of the first infrared light received by the imaging part, and combines the first image and the second image.

In an image forming apparatus according to a third aspect of the present invention, in the first or second aspect described above, the imaging part may include a splitting unit that splits the light from the observation target irradiated with the excitation light into a component of the first infrared light and a component of the second infrared light.

In an image forming apparatus according to a fourth aspect of the present invention, in the third aspect described above, the splitting unit may further split the light from the observation target irradiated with the excitation light into light having a visible light wavelength.

In an image forming apparatus according to a fifth aspect of the present invention, in the fourth aspect described above, the image processing unit may further include a visible light image processing unit that generates a visible light image corresponding to the light having a visible light wavelength received by the imaging part, and the visible light image may be added to the image obtained by combining the first image and the second image at a specific ratio to generate a composite image.

In an image forming apparatus according to a sixth aspect of the present invention, in the fourth or fifth aspect described above, the imaging part may include an optical system that corrects a focus shift of the light from the observation target irradiated with the excitation light, including from visible light to short wavelength infrared light.

In an image forming apparatus according to a seventh aspect of the present invention, in any one of the first to sixth aspects described above, the imaging part may include a filter that cuts the excitation light from the light from the observation target irradiated with the excitation light.

In an image forming apparatus according to an eighth aspect of the present invention, in any one of the first to seventh aspects described above, the image processing unit may generate the composite image in which a brightness signal of a region outside the specific region in the first image is corrected to zero.

As described above, the present invention relates to an observation device capable of acquiring a composite image in which a short wavelength-side near-infrared light image and a short wavelength infrared light image of an object containing a fluorescent substance are combined. According to the above-described embodiments of the present invention, it is possible to form an image including the height of the contrast according to the first image and the image density according to the second image, and it is possible to more precisely specify a target region in a biological tissue.

According to the present invention, it is possible to clearly present a result of an examination using fluorescence in a living body. Therefore, the present invention is expected to contribute to the achievement of the sustainable development goals (SDGs) for ensuring healthy life and promoting welfare.

What is claimed is:

1. An image forming apparatus comprising:
an excitation light source that irradiates an observation target with excitation light;
an imaging part that receives first infrared light and second infrared light split from light from the observation target irradiated with the excitation light, the first infrared light including light having a wavelength in a short wavelength infrared region, and the second infrared light including light having a wavelength in a wavelength region on a shorter wavelength side than the short wavelength infrared region; and
an image processing unit that generates a composite image by combining a first image and a second image, the first image indicating a boundary of a specific region corresponding to the first infrared light received by the imaging part, and the second image including a specific region having an image density corresponding to the second infrared light received by the imaging part.

2. The image forming apparatus according to claim 1, wherein the image processing unit includes an infrared image combining unit that generates the first image indicating a contour of an image of the specific region by binarizing an amount of the first infrared light received by the imaging part, and combines the first image and the second image.

3. The image forming apparatus according to claim 1, wherein the imaging part includes a splitting unit that splits the light from the observation target irradiated with the excitation light into a component of the first infrared light and a component of the second infrared light.

4. The image forming apparatus according to claim 3, wherein the splitting unit further splits the light from the observation target irradiated with the excitation light into light having a visible light wavelength.

5. The image forming apparatus according to claim 4, wherein the image processing unit further includes a visible light image processing unit that generates a visible light image corresponding to the light having a visible light wavelength received by the imaging part, and
the visible light image is added to the image obtained by combining the first image and the second image at a specific ratio to generate a composite image.

6. The image forming apparatus according to claim 5, wherein the imaging part includes an optical system that corrects a focus shift of the light from the observation target irradiated with the excitation light in a range of from visible light to short wavelength infrared light.

7. The image forming apparatus according to claim 1, wherein the imaging part includes a filter that cuts the excitation light from the light from the observation target irradiated with the excitation light.

8. The image forming apparatus according to claim 1, wherein the image processing unit generates the composite image in which a brightness signal of a region outside the specific region in the first image is corrected to zero.
